(19) United States
(12) Patent Application Publication
(10) Pub. No.: US 2015/0027940 A1
(43) Pub. Date: Jan. 29, 2015

(54) PORTABLE STERILIZATION APPARATUS

(71) Applicants: YOUNG HUN LEE, CHUNCHEON-SI (KR); JUNG RYUL LEE, CHUNCHEON-SI (KR); PLUSE CO., LTD., CHUNCHEON-SI (KR)

(72) Inventors: Young Hun Lee, Chuncheon-si (KR); Jung Ryul Lee, Chuncheon-si (KR)

(21) Appl. No.: 13/881,733

(22) PCT Filed: Aug. 2, 2012
(86) PCT No.: PCT/KR12/06155

§ 371 Date: Apr. 26, 2013

(30) Foreign Application Priority Data
Apr. 17, 2012 (KR) 10-2012-0039652

(51) Int. Cl. C02F 1/50; C02F 1/461

(52) U.S. Cl. CPC C02F 1/50; C02F 1/46104; C02F 2201/461; C02F 2305/023
USPC 210/192

(57) ABSTRACT

Disclosed herein is a portable sterilization apparatus. The portable sterilization apparatus includes: a case; a power supply disposed inside the case; a power supply disposed inside the case; a hydroxyl radical generator electrically connected to the power supply and exposed outside from the case; and a switch unit disposed inside the case and selectively supplying electric power from the power supply to the hydroxyl radical generator.

### FIELD OF TECHNOLOGY

The following relates to a portable sterilization apparatus, and more particularly, to a portable sterilization apparatus which can be miniaturized by maximizing generation of hydroxyl radicals and provides portability for convenient use.

### BACKGROUND

Generally, a hydroxyl radical generator is placed in water of a humidifier, an air cleaner, or the like, and generates low temperature plasma with electric energy through induction of electric discharge between a negative electrode and a positive electrode by receiving relatively stable low voltage direct current (DC) power.

Hydroxyl radicals are the neutral form of the hydroxide ion, can be produced in the fourth state of matter known as plasma and are a natural occurring substance having sterilization, disinfection and decomposition capabilities at least twice stronger than ozone and chlorine, without being toxic or hazardous to human health.

Low-temperature plasma produces hydroxyl radicals (OH—) in water and destroys germs, algae, etc., thereby preventing secondary pollution due to polluted water by preventing germs from propagating in water.

Korean Patent Publication No. 10-2010-0084735A (published on Jul. 28, 2010) discloses a “portable hydroxyl radical generator”.

A typical hydroxyl radical generator is attached to the bottom of a water tank or product and thus has limited applicability. In addition, such a hydroxyl radical generator has low discharge efficiency due to a mesh-shaped electrode, and requires soldering or spot-welding between the electrode and a power supply line, causing various problems, such as volume increase, material waste, and waterproofing difficulty. Therefore, there is a need for an improved hydroxyl radical generator.

### SUMMARY

### Technical Problem

It is an aspect of the present invention to provide a portable sterilization apparatus, which has improved discharge efficiency and a small volume through a stacked electrode structure, and provides portability through use of charged electric power.

It is another aspect of the present invention to provide a portable sterilization apparatus, which includes a power supply integrally formed with a plurality of electrodes arranged in a stacked structure, thereby allowing easy manufacture of various capacities, and which has a convenient connection structure between the electrodes and the power supply to facilitate assembly, thereby improving productivity.

### Technical Solution

In accordance with one aspect of the present invention, a portable sterilization apparatus incudes: a case; a power supply disposed inside the case; a hydroxyl radical generator electrically connected to the power supply and exposed outside the case; and a switch unit disposed inside the case and selectively supplying electric power from the power supply to the hydroxyl radical generator.

The case may be provided with an exposure cap to expose the hydroxyl radical generator to an outside; and the exposure cap may be detachable from the case.

The hydroxyl radical generator may include: a plurality of positive electrodes electrically connected to the power supply; a plurality of negative electrodes electrically connected to the power supply; and an electrode guide allowing the plurality of negative electrodes and the plurality of positive electrodes to be alternately stacked one above another in a separated state from each other, and electrically connecting the plurality of negative electrodes to each other while electrically connecting the plurality of positive electrodes to each other.

The electrode guide may include: a pair of electric bars arranged in parallel to allow the plurality of positive electrodes and the plurality of negative electrodes to be electrically connected to the power supply; connecting members each fitted to the electric bar and connecting the positive electrodes or the negative electrodes to each other such that the positive electrodes are electrically connected only to each other thereby or the negative electrodes are electrically connected only to each other thereby; and an insulating member disposed on a circumferential surface of each of the connecting members to prevent electrical conduction between the positive electrodes and the negative electrodes and to separate the positive electrodes and the negatives electrode from each other.

Each of the positive electrodes and the negative electrodes may include a contact hole and a non-contact hole separated from each other. Here, the contact holes and the non-contact holes of electrodes having the same polarity may be collinearly arranged, and the contact holes and the non-contact holes of electrodes having different polarities are arranged opposite each other.

The contact hole may have a smaller diameter than the connecting member; and the non-contact hole may have a diameter equal to or greater than that of the insulating member.

Each of the connecting members may be formed on opposite surfaces thereof with a protrusion and a groove corresponding to each other, respectively, and the connecting members may be stacked and connected by the protrusions and the grooves.

Each of the positive electrodes and the negative electrodes may have a plate shape, and may include electrode protrusions formed on opposite sides thereof .

The switch unit may include a water level sensor detecting contact with water and a control circuit controlling the power supply to supply electric power to the hydroxyl radical generator in response to a signal from the water level sensor.

The water level sensor may be separated a predetermined distance from the hydroxyl radical generator to allow smooth discharge of water from the case.

### Advantageous Effects

According to the invention, the portable sterilization apparatus includes a plurality of positive electrodes and a plurality of negative electrodes alternately stacked to improve discharge efficiency, and each of the positive electrodes and the negative electrodes is formed with an electrode protrusion to maximize discharge efficiency in the same area, thereby enabling volume reduction of finished products.

In addition, the portable sterilization apparatus according to the invention enables size reduction of finished products and uses charged electric power of the power supply, thereby providing portability.

Further, the portable sterilization apparatus according to the invention is provided with an electrode guide to improve the stacked structure of the plurality of positive and negative electrodes, and thus can eliminate an existing connection structure based on soldering or the like, thereby improving assembly efficiency and productivity.

Furthermore, the portable sterilization apparatus according to the invention employs an exposure cap detachable from the case to thereby facilitate replacement and maintenance of the hydroxyl radical generator, and a switch unit for supplying electric power according to the presence of water to thereby prevent malfunction and provide operation convenience.

### BRIEF DESCRIPTION

The above and other aspects, features and advantages of the present invention will become apparent from the following description of embodiments given in conjunction with the accompanying drawings, in which:

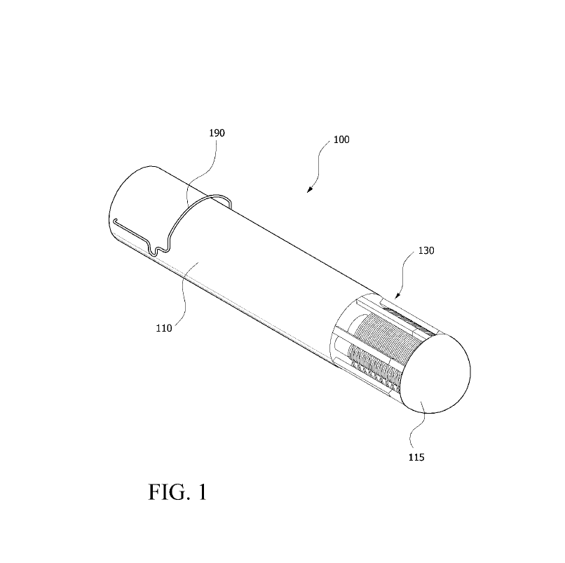

FIG. 1 is a perspective view of a portable sterilization apparatus according to one embodiment of the present invention;

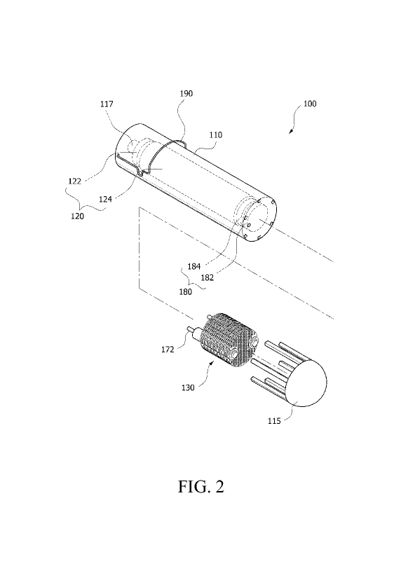

FIG. 2 is an exploded perspective view of the portable sterilization apparatus according to the embodiment of the present invention;

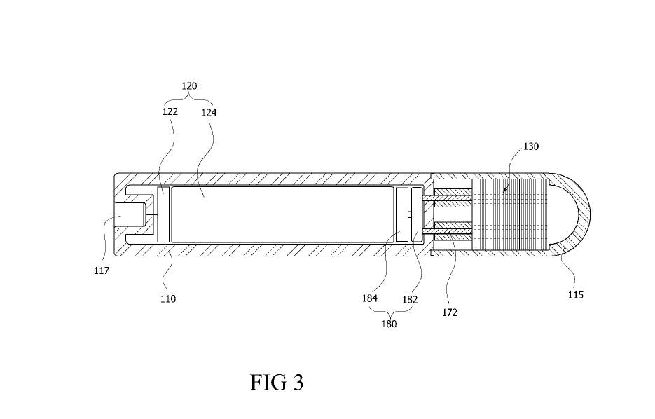

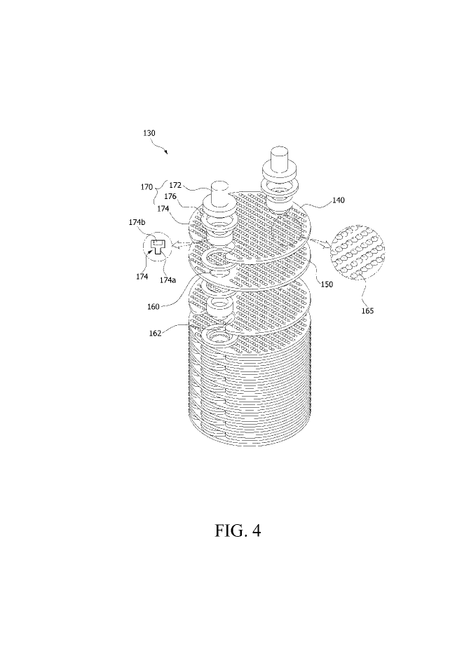

FIG. 3 is a sectional view of the portable sterilization apparatus according to the embodiment of the present invention; FIG. 4 is an exploded perspective view of a hydroxyl radical generator in the portable sterilization apparatus according to the embodiment of the present invention;

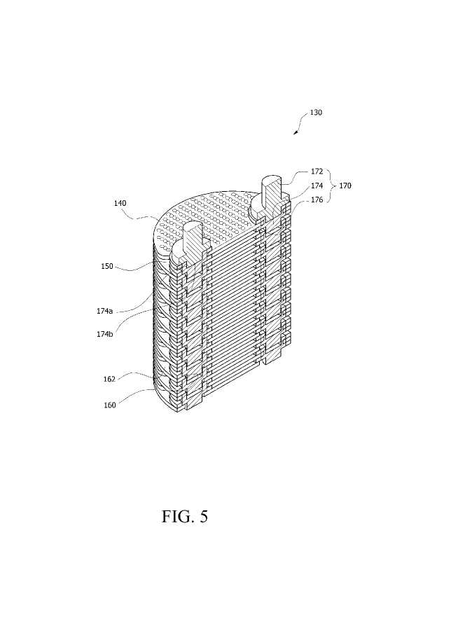

FIG. 5 is a sectional view of the hydroxyl radical generator in the portable sterilization apparatus according to the embodiment of the present invention;

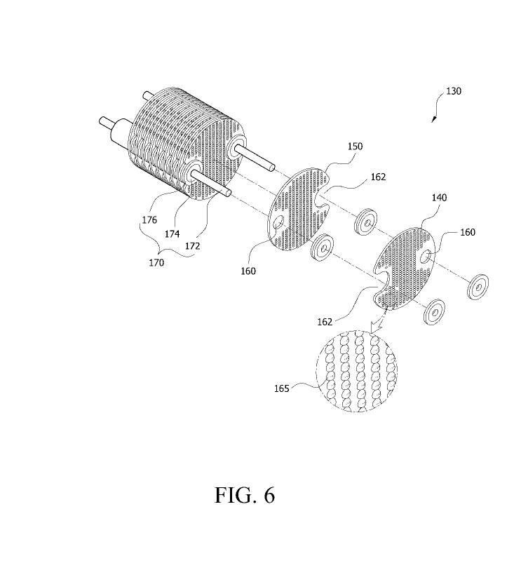

FIG. 6 is an exploded perspective view of modification of the hydroxyl radical generator in the portable sterilization apparatus according to the embodiment of the present invention;

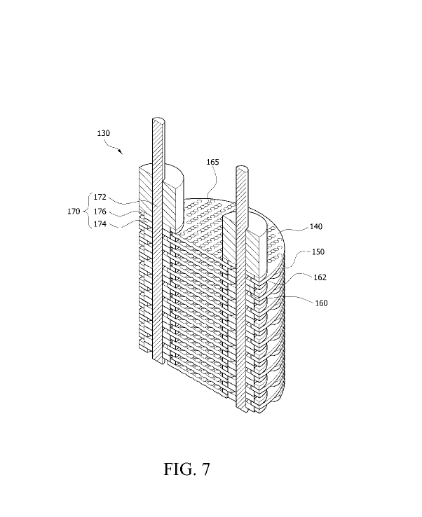

FIG. 7 is a sectional view of modification of the hydroxyl radical generator in the portable sterilization apparatus according to the embodiment of the present invention; and

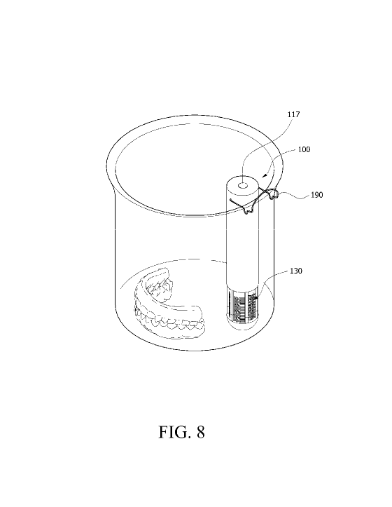

FIG. 8 is a diagram of the portable sterilization apparatus according to the embodiment of the present invention in use.

### DETAILED DESCRIPTION

Embodiments of the invention will now be described in detail with reference to the accompanying drawings. It should be noted that the drawings are not to precise scale and may be exaggerated in thickness of lines or size of components for descriptive convenience and clarity. Furthermore, the terms used herein are defined by taking functions of the present disclosure into account and can be changed according to user or operator's custom or intention. Therefore, the terms should be defined according to the overall disclosure set forth herein.

FIG. 1 is a perspective view of a portable sterilization apparatus according to one embodiment of the present invention, FIG. 2 is an exploded perspective view of the portable sterilization apparatus according to the embodiment of the invention, FIG. 3 is a sectional view of the portable sterilization apparatus according to the embodiment of the invention, FIG. 4 is an exploded perspective view of a hydroxyl radical generator in the portable sterilization apparatus according to the embodiment of the invention, and FIG. 5 is a sectional view of the hydroxyl radical generator in the portable sterilization apparatus according to the embodiment of the invention.

Referring to FIGS. 1 to 5, a portable sterilization apparatus 100 according to one embodiment of the present invention includes a case 110, a power supply 120, a hydroxyl radical generator 130, and a switch unit 180.

The case 110 is made of a plastic material, has a space that receives the power supply 120 and the switch unit 180 therein, and is sealed for waterproofing. Further, the case is provided at a rear side thereof with a rotatable holding member 190.

The case 110 includes an exposure cap 115. The exposure cap 115 has an open circumferential surface through which the hydroxyl radical generator 130 is exposed to the outside, and is detachable from the case 110. That is, the exposure cap 115 is detachably coupled to a leading end of the case 110 by various coupling methods, such as hook coupling, screw coupling, and the like.

The power supply 120 is placed inside the case 110 and connected to an external power source to be charged thereby. The power supply 120 includes a charge circuit 122 which converts external alternating current (AC) power into direct current (DC) power, and a battery 124 which is charged with DC power output from the charge circuit 122.

The case 110 is provided at the rear side thereof with a socket 117 through which the external power source can be connected to the charge circuit 122. Here, it should be noted that electric power to be supplied to the battery 124 is relatively safe direct current (DC) in order to prevent accidents such as electric shock or the like, and that a switching mode power supply (SMPS) having over-current protection is used.

Although the power supply 120 is illustrated as including the charge circuit 122 and the battery 124 charged with external power in this embodiment, the present invention is not limited thereto. Alternatively, a replaceable primary battery may be used as the power supply 120.

The hydroxyl radical generator 130 is electrically connected to the power supply 120 and exposed to the outside from the case 110. That is, the hydroxyl radical generator 130 generates hydroxyl radicals through electrolysis of water when submerged into water and receiving electric power from the power supply 120. For this purpose, the hydroxyl radical generator 130 is disposed inside the exposure cap 115 detachable from the case 110. That is, the hydroxyl radical generator 130 contacts water through an exposed section of the exposure cap 115.

The hydroxyl radical generator 130 includes a plurality of positive electrodes 140 electrically connected to the power supply 120, a plurality of negative electrodes 150 electrically connected to the power supply 120, and an electrode guide 170 on which the plurality of negative electrodes 150 and the plurality of positive electrodes 140 are separated from each other and alternately stacked one above another, and which electrically connects the plural negative electrodes 150 only to each other and the plural positive electrodes 140 only to each other.

The positive electrodes 140 and the negative electrodes 150 are made of an electrically conductive metal. For example, the positive electrodes 140 and the negative electrodes 150 may be formed by vacuum deposition or plating of a platinum-based metal on an SUS304 or titanium plate.

As shown in FIG. 4 or 5, the electrode guide 170 includes a pair of electric bars 172 arranged in parallel such that each of the positive and negative electrodes 140, 150 can be electrically connected to the power supply 120 thereby, connecting members 174 each connected to the electric bar 172 connecting the positive electrodes 140 and the negative electrodes 150 such that the positive electrodes 140 are electrically connected only to each other via the connecting member and the negative electrodes 150 are electrically connected only to each other via the other connecting member, and an insulating member 176 disposed on a circumferential surface of each of the connecting members 174 to prevent electrical conduction between the positive electrodes 140 and the negative electrodes 150 while separating the positive electrodes 140 and the negative electrodes 150 from each other.

Here, each of the connecting members 174 allows only electrodes having the same polarity to be electrically connected to each other, and is formed by plating or vacuum deposition of a platinum-based metal onto titanium or conductive metal. Further, the insulating member 176 prevents electrical conduction and is formed of an insulating material, such as polyvinyl chloride (PVC), acrylonitrile butadiene styrene (ABS), polycarbonate (PC), etc.

Each of the connecting members 174 is formed on opposite surfaces thereof with a protrusion 174a and a groove 174b corresponding to each other, respectively, whereby the connecting members 174 can be stacked and connected by the protrusions 174a and the grooves 174b. That is, the electric bar 172 is inserted into and electrically connected to the groove 174b of the outermost connecting member 174, and electrical conduction through the connecting member 174 is permitted by coupling between the protrusion 174a and the groove 174b.

Here, the protrusion 174a and the groove 174b may be press-fitted to each other to maintain a stacked state. Alternatively, the protrusion 174a and the groove 174b may be prevented from being separated by a friction protrusion (not shown) and a friction groove (not shown) corresponding to an external diameter of the protrusion 174a and an internal diameter of the groove 174b, respectively.

Further, the insulating member 176 has a ring shape and has an inner diameter equal to an outer diameter of the connecting member 174 such that the connecting member 174 can be accommodated within the insulating member 176.

Further, each of the connecting member 174 and the insulating member 176 has a ring shape, and the electric bar 172 passes through the center of the connecting member 174. Here, the inner diameter of the insulating member 176 is equal to the outer diameter of the connecting member 174, whereby the connecting member 174 can be accommodated within the insulating member 176.

Further, each of the positive electrodes 140 and the negative electrodes 150 is provided at opposite sides thereof with a contact hole 160 and a non-contact hole 162, which are separated from each other, respectively. The contact hole 160 has a smaller diameter than the connecting member 174, and the non-contact hole 162 has a diameter equal to or greater than that of the insulating member 176. That is, top and bottom surfaces of the electrode around the contact hole 160 are in surface-contact with the connecting member 174 and thus allow electrical conduction therebetween. The insulating member 176 is received in the non-contact hole 162 and prevents contact between the electrode and the connecting member 174, thereby preventing electrical conduction therebetween.

Specifically, the contact hole 160 and the non-contact hole 162 are formed at opposite sides of each electrode and separated from each other. At one side, the contact holes 160 of the positive electrodes 140 and the non-contact holes 162 of the negative electrodes 150 are collinearly aligned. At the other side, the non-contact holes 162 of the positive electrodes 140 and the contact holes 160 of the negative electrodes 150 are collinearly aligned. Further, the connecting member 174 and the insulating member 176 are fitted to each other.

In this state, when the electrodes are compressed to come into close-contact with each other, the insulating member 176 having a large diameter is interposed between the positive and negative electrodes 140, 150 alternately stacked, so that the positive electrodes 140 and the negative electrodes 150 can be separated a predetermined distance from each other, and the connecting member 174 contacts not the non-contact hole 162 but the contact hole 160, thereby allowing electrical conduction only through electrodes having the same polarity.

In this way, only the electrodes having the same polarity are electrified, and the positive electrodes 140 and the negative electrodes 150 are alternately stacked by the electric bars 172, whereby the hydroxyl radical generator 130 can be realized in the form of a cartridge.

FIG. 6 is an exploded perspective view of modification of the hydroxyl radical generator in the portable sterilization apparatus according to the embodiment of the present invention, and FIG. 7 is a sectional view of modification of the hydroxyl radical generator in the portable sterilization apparatus according to the embodiment of the present invention.

Referring to FIGS. 6 and 7, the electric bars 172 may be elongated to pass through the positive electrodes 140 and the negative electrodes 150, respectively. Here, each of the connecting members 174 and the insulating member 176 may have a ring shape, each of the electric bars 172 may extend through the center of the connecting member 174, and each of the connecting members 174 electrically connects electrodes having the same polarity to each other through connection to the contact holes 160 and disconnection from the non-contact holes 162.

Each of the positive electrodes 140 and the negative electrodes 150 has a plate shape, and includes electrode protrusions 165 formed on opposite sides thereof The electrode protrusions 165 improve discharge efficiency per unit area, whereby discharge can be efficiently induced in a small area of the positive and negative electrodes 140, 150.

Further, the switch unit 180 is placed inside the case 110 and selectively supplies electric power from the power supply 120 to the hydroxyl radial generator 130 according to the presence of water. The switch unit 180 includes a water level sensor 182 which detects contact with water, and a control circuit 184 which controls electric power to be supplied to the hydroxyl radical generator 130 in response to signals from the water level sensor 182. That is, when the water level sensor 182 of the switch unit 180 is submerged in water, the hydroxyl radical generator 130 automatically operates, and when the water level sensor 182 of the switch unit 180 is raised above water, the hydroxyl radical generator 130 automatically stops operation.

The water level sensor 182 is made of a capacitive semiconductor and is provided inside the case 110 to prevent malfunction.

In this embodiment, the switch unit 180 operates in response to detection of water by the water level sensor 182, without being limited thereto. Alternatively, the switch unit 180 may be realized by various ways. For example, the switch unit 180 may include a button switch or the like, which is disposed on a circumferential surface of the case 110 and outputs an operation signal.

Next, operation of the portable sterilization apparatus according to this embodiment of the present invention will be described.

First, when an external power source is connected to the socket 117 on the rear side of the case 110, the charge circuit 122 charges the battery 124 with DC power.

In this way, the power supply 120 of the sterilization apparatus according to this embodiment may be charged with external power, whereby the sterilization apparatus can be conveniently used outside.

Further, the hydroxyl radical generator 130 exposed outside from the case 110 has a structure in which the plurality of positive electrodes 140 and the plurality of negative electrodes 150 are separated from each other and alternately stacked one above another, thereby enabling size reduction of finished products. In addition, there is no limit to a stacking height of the positive electrodes 140 and the negative electrodes 150, thereby allowing the portable sterilization apparatus to be manufactured in various capacities.

As shown in FIGS. 4 and 5, the hydroxyl radical generator 130 includes the plurality of positive electrodes 140 and the plurality of negative electrodes 150, which are separated a predetermined distance from each other by the electrode guide 170, which electrically connects only electrodes having the same polarity to each other for electrical conduction therethrough. In more detail, each electrode is formed with the contact hole 160 and the non-contact hole 162. With the structure in which the contact hole 160 is electrified by the connecting member 174 but the non-contact hole 162 is not electrified by the insulating member 176, electrodes having the same polarity are electrified and electrodes having different polarities are not electrified.

The aforementioned operation can be achieved since the contact holes 160 and the non-contact holes 162 of the electrodes having the same polarity are collinearly aligned and the contact holes 160 and the non-contact holes 162 of the electrodes having different polarities are opposite each other.

This structure eliminates a molding process that is used in assembly of a general hydroxyl radical generating module and employs epoxy, urethane, rubber, and the like to provide a waterproofing structure after connection via soldering, spot-welding or the like, thereby improving assembly efficiency. Further, in this structure, the electrodes eliminate the need for the waterproofing structure and can generate a considerably large amount of hydroxyl radicals, thereby improving electrode efficiency while reducing the size of finished products. Further, the apparatus can prevent corrosion of a soldered zone or a spot-welded zone due to defective waterproofing, or can prevent leakage of contaminants (lead, rust, etc.) to the outside.

Further, the electrodes may be stacked one above another by the protrusions 174a and the grooves 174b of the connecting members 174 press-fitted to each other, thereby improving assembly efficiency. Here, the number of electrodes stacked one above another may be increased or decreased according to desired capacity.

Referring to FIG. 8, the portable sterilization apparatus 100 according to this embodiment may be used as follows. After placing dentures as a sterilizing target in a container filled with water, the leading end of the case 110 is submerged into the water.

Although dentures are described as the sterilizing target in this embodiment, the invention is not limited thereto. Alternatively, the apparatus 100 may be used for sterilization of other objects.

Dentures can be stored in a cup or other suitable container filled with water when not in use. In this case, however, germs in the dentures or water proliferate and further contaminate the dentures over time. When a user wears such dentures infected with germs, the user can also be infected with germs. To prevent this problem, the portable sterilization apparatus 100 is submerged into water.

The rotatable holding member 190 is provided to the rear side of the case 110, whereby the portable sterilization apparatus 100 can be fixed to the rim of the container via the holding member.

The switch unit 180 is disposed inside the case 110. When the water level sensor 182 of the switch unit 180 detects water, the water level sensor 182 sends a signal to the control circuit 184, which in turn controls the power supply 120 to supply electric power to the hydroxyl radical generator 130, thereby generating hydroxyl radicals.

The hydroxyl radicals in water provide sterilizing power for 1 to 2 hours. Thus, generation of hydroxyl radicals is controlled by repeating operation/stop for a predetermined duration such that sterilization can be realized indefinitely.

That is, the portable sterilization apparatus 100 may automatically operate when submerged in water, and may automatically stop when raised above water. Since the water level sensor 182 is operated by water, the water level sensor 182 of the switch unit 180 is separated a predetermined distance from the hydroxyl radical generator 130 to ensure smooth discharge of water from the case in order to prevent operation failure.

Upon supply of electric power from the power supply 120, hydroxyl radicals are generated through induction of electric discharge of the positive electrodes 140 and the negative electrodes 150, thereby preventing infection or disease due to germs.

In this embodiment, each of the positive and negative electrodes 140, 150 is formed with the electrode protrusions in opposite directions thereof, so that a unit area can be enlarged, thereby maximizing efficiency of electric discharge.

Further, the hydroxyl radical generator 130 is realized in the form of a cartridge and is accommodated in the exposure cap 115. Here, since the exposure cap 115 is detachable from the case, the hydroxyl radical generator 130 can be easily replaced, thereby facilitating maintenance.

As such, in the portable sterilization apparatus according to the invention, the plurality of electrodes is stacked one above another and the electrode protrusions are formed on each of the electrodes to maximize the efficiency of electric discharge, thereby improving sterilizing power.

In addition, in the portable sterilization apparatus according to the invention, a power supply is integrally formed with the plurality of electrodes, which are arranged in a stacked structure, thereby allowing manufacture according to desired capacities. Further, the portable sterilization apparatus according to the invention has a convenient connection structure between the electrodes and the power supply to facilitate assembly, thereby improving productivity.

Although some embodiments have been described herein with reference to the accompanying drawings, it will be understood by those skilled in the art that these embodiments are provided for illustration only, and various modifications, changes, alterations and equivalent embodiments can be made without departing from the scope of the invention. Therefore, the scope and sprit of the invention should be defined only by the accompanying claims and equivalents thereof

1. A portable sterilization apparatus comprising:
a case;
a power supply disposed inside the case;
a hydroxyl radical generator electrically connected to the power supply and exposed outside from the case; and
a switch unit disposed inside the case and selectively supplying electric power from the power supply to the hydroxyl radical generator.

2. The portable sterilization apparatus according to claim 1, wherein the case is provided with an exposure cap to expose the hydroxyl radical generator to an outside; and the exposure cap is detachable from the case.

3. The portable sterilization apparatus according to claim 1, wherein the hydroxyl radical generator comprises:
a plurality of positive electrodes electrically connected to the power supply;
a plurality of negative electrodes electrically connected to the power supply; and
an electrode guide allowing the plurality of negative electrodes and the plurality of positive electrodes to be alternately stacked one above another in a separated state, and electrically connecting the plurality of negative electrodes to each other while electrically connecting the plurality of positive electrodes to each other.

4. The portable sterilization apparatus according to claim 3, wherein the electrode guide comprises:
a pair of electric bars passing through the plurality of positive electrodes and the plurality of negative electrodes, respectively, the pair of electric bars being electrically connected to the power supply and arranged in parallel;
connecting members each fitted to the electric bar and connecting the positive electrodes or the negative electrodes to each other such that the positive electrodes are electrically connected only to each other thereby or the negative electrodes are electrically connected only to each other thereby; and
an insulating member disposed on a circumferential surface of each of the connecting members to prevent electrical conduction between the positive electrodes and the negative electrodes and to separate the positive electrodes and the negatives electrode from each other.

5. The portable sterilization apparatus according to claim 4, wherein each of the positive electrodes and the negative electrodes comprises a contact hole and a non-contact hole, through which the electric bar passes and which are separated from each other; and the contact holes and the non-contact holes of electrodes having the same polarity are collinearly arranged, and the contact holes and the non-contact holes of electrodes having different polarities are arranged opposite each other.

6. The portable sterilization apparatus according to claim 5, wherein the contact hole has a smaller diameter than the connecting member; and the non-contact hole has a diameter equal to or greater than that of the insulating member.

7. The portable sterilization apparatus according to claim 4, wherein each of the connecting members is formed on opposite surfaces thereof with a protrusion and a groove corresponding to each other, respectively, the connecting members being stacked and connected by the protrusions and the grooves.

8. The portable sterilization apparatus according to claim 3, wherein each of the positive electrodes and the negative electrodes has a plate shape and includes electrode protrusions formed on opposite surfaces thereof

9. The portable sterilization apparatus according to claim 1, wherein the switch unit comprises a water level sensor detecting contact with water; and a control circuit controlling the power supply to supply electric power to the hydroxyl radical generator in response to a signal from the water level sensor.

10. The portable sterilization apparatus according to claim 9, wherein the water level sensor is separated a predetermined distance from the hydroxyl radical generator to allow smooth discharge of water from the case.
